(19) United States
(12) Patent Application Publication
(10) Pub. No.: US 2026/0066086 A1
(43) Pub. Date: Mar. 5, 2026

(54) CONVOLUTIONAL NEURAL NETWORK CLASSIFICATION OF PRETREATMENT BIOPSIES

(71) Applicant: Washington University, St. Louis, MO (US)

(72) Inventors: Michael Waters, St. Louis, MO (US); Jin Zhang, St. Louis, MO (US); Hyun Kim, St. Louis, MO (US)

(21) Appl. No.: 19/105,243

(22) PCT Filed: Oct. 9, 2023
(86) PCT No.: PCT/US2023/076361

§ 371 Date: Feb. 20, 2025

Related U.S. Application Data
(60) Provisional application No. 63/378,698, filed on Oct. 7, 2022.

(51) Int. Cl. G16H 20/40; G06N 3/0464; G16H 30/20

(52) U.S. Cl. CPC G16H 20/40; G06N 3/0464; G16H 30/20

(57) ABSTRACT

A method and apparatus are provided for pretreatment prediction system comprising display device a computing device comprising a processor and a memory, the memory storing instructions that, when executed by the processor cause the processor to receive at least one image of a pre-treatment biopsy from a patient process the at least one image using a trained convolutional neural network (CNN) display on the display device, a prediction of a response of the patient to a treatment base on a result of the processing of the at least one image using the trained CNN.

### I. RELATED APPLICATIONS

The present application claims priority to U.S. Provisional Patent Application No. 63/378,698 entitled “Systems and Methods Using Convolutional Neural Networks for Pretreatment Prediction of Response to a Treatment,” filed Oct. 7, 2022, the entirety of which is incorporated by reference herein for all purposes.

### II. FIELD OF THE DISCLOSURE

The present disclosure relates generally to computer networks and associated processes, and more particularly, to using computer vision related technologies to facilitate a prognosis and response to therapy for patients afflicted with certain types of cancer.

### III. BACKGROUND

A nonoperative treatment with short course radiation (SCRT) followed by chemotherapy provides a promising tool to treat rectal cancer. For those who completely respond to the protocol, the treatment offers high efficacy while maintaining rectal function. However, a certain percentage of patients will incompletely respond to the treatment. For them, the physical and financial hardships associated with chemotherapy may retroactively be perceived as being in vain.

### IV. SUMMARY OF THE DISCLOSURE

According to an embodiment, an apparatus is provided that comprises a pretreatment prediction system comprising display device a computing device comprising a processor and a memory, the memory storing instructions that, when executed by the processor cause the processor to receive at least one image of a pre-treatment biopsy from a patient process the at least one image using a trained convolutional neural network (CNN) display on the display device, a prediction of a response of the patient to a treatment base on a result of the processing of the at least one image using the trained CNN.

According to another embodiment, a method of predicting a treatment outcome, the method comprising receiving at least one image of a pre-treatment biopsy from a patient; processing the at least one image using a trained CNN, and displaying, on a display device, a prediction of a response of the patient to a treatment base on a result of the processing of the at least one image using the trained CNN.

According to another embodiment, a computer readable storage medium includes instructions that when executed by a processor cause the processor to receive at least one image of a pre-treatment biopsy from a patient; process the at least one image using a trained CNN, and display, on a display device, a prediction of a response of the patient to a treatment base on a result of the processing of the at least one image using the trained CNN.

Implementations may identify patients likely to experience a clinical complete response to radiation and/or chemotherapy and/or immune therapy to prevent overtreatment with radiation and/or chemotherapy and/or immune therapy in early-stage patients and progression to metastatic disease in those unlikely to achieve complete response.

Features and other benefits that characterize embodiments are set forth in the claims annexed hereto and forming a further part hereof. However, for a better understanding of the embodiments, and of the advantages and objectives attained through their use, reference should be made to the Drawings and to the accompanying descriptive matter.

### V. BRIEF DESCRIPTION OF THE DRAWINGS

FIG. 1 is a block diagram of an embodiment of a system configured to use a convolutional neural network for the pretreatment prediction of a response to a treatment. The system depicts aspects of training data in addition to hardware and software for analyzing an image data to make a determination of the likely success of a patient's proposed treatment;

FIG. 2 is block diagram of an embodiment of a system used to train a convolutional neural network to classify image data according to its likelihood of being associated with a patient who will or will not respond to a given therapy;

FIG. 3 shows a series of images that include image data associated with complete responders, as well as image data associated with incomplete responders;

FIG. 4 generally illustrates a data processing apparatus configured to train and assess predictions using image data;

FIG. 5 is a flowchart of an embodiment of a method of predicting using a convolutional neural network of pre-treatment histopathological images to identify patients who will experience a complete response to at least one of: a radiation, a chemotherapy, or an immune therapy treatment; and

FIG. 6 is a flowchart of an embodiment of a method of training a convolutional neural network to classify image data according to its likelihood of being associated with a patient who will or will not respond to a given therapy.

### VI. DETAILED DESCRIPTION

An implementation may use a convolutional neural network of pre-treatment histopathological images to identify patients who will experience a complete response to short-course consolidation radiotherapy and consolidation chemotherapy. More particularly, an embodiment of a system may identify patients likely to experience a complete response to at least one of: a radiation, a chemotherapy, or an immune therapy treatment. These treatment courses may maximize tumor down staging prior to surgical resection or be used to achieve clinical complete response in order for watch and wait or nonoperative management to be pursued. In one instance, an implementation may reduce overtreatment with chemotherapy and the risk of metastatic progression for incomplete responders.

While techniques discussed herein are described in greater detail in terms of treating rectal cancer in particular, one skilled in the art will recognize that the underlying methods may be used in the treatment of other cancers and diseases. Illustrative treatment protocols may include chemoradiation followed by chemotherapy, and chemotherapy followed by chemoradiation. Other types of treatment contemplated in this application include chemotherapy followed by SCRT, or any combination of the above, to include immune therapy as well. Embodiments of the system are contemplated that include either of short or long course chemoradiotherapy.

A particular embodiment may generate or receive image files of hematoxylin and eosin-stained pre-treatment rectal adenocarcinoma biopsies. One skilled in the art will recognize that other implementations use other types of stains. Such biopsies may be acquired using a microscopy software suite, for example. In one instance, the image files may be exported in compressed image files.

The image files may be automatically segmented using grid software tools. For example, grid images comprised largely of whitespace may be removed. The remaining grid images may be exported for modeling and/or other evaluation in jpeg or another image format that preserves micron scale pixel resolution. For instance, the remaining grid images may be used as training datasets in connection with the convolutional neural network classifier. Such image files may be received from multiple sources over an extended period of time to increase the efficacy of the training. Illustrative such training includes programmatically evaluating images correlated to complete responders and partial responders.

In addition to the grid segmentation processes described herein, implementations may include other types of preprocessing actions. For instance, a particular embodiment may remove whitespace from received or segmented images, while another or the same embodiment may rescale tissue features within an image or segmented image. Other embodiments may flip and rotate the image data to facilitate automatic identification.

The classifying processes at the convolutional neural network classifier may be fine-tuned using mathematical weighting to predict biological behavior or responsiveness to therapy in rectal cancer. Such weighting may be applied to different features of tissue captured in the images adjusted automatically according to the accuracy of the prediction. Illustrative features of tissue captured in the images may include size, density, shape, proximity to one another, color, among other tissue characteristics identifiable within an image. As such, training may include program code processing empirical data with different weighting scenarios. The results of the scenarios may be correlated with treatment results to determine the accuracy of the predictions. The weighting combinations resulting with more accurate predictions may be used to iteratively refine the classification processes. Put another way, nodal weights are updated based on training from histopathological slide images, thus constantly improving the accuracy of the classifier.

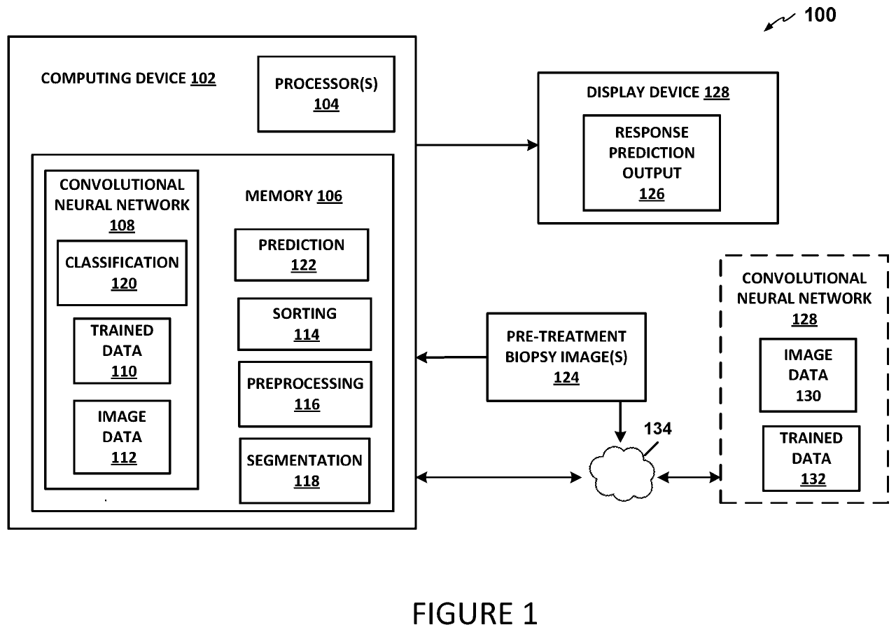

FIG. 1 is a block diagram of an embodiment of a system 100 configured to use a convolutional neural network for the pretreatment prediction of a response to a treatment. The system 100 depicts aspects of training data in addition to hardware and software for analyzing image data to make a determination of the likely success of a patient's proposed treatment.

Turning more particularly to the FIG. 1, the system 100 includes a computing device 102 comprising one or more processors 104. While depicted in a single block 104 in FIG. 1, other implementations may include multiple processors distributed locally and/or remotely throughout multiple networked or electronically linked topologies.

The computing device 102 of the embodiment of the system 100 may additionally include a memory 106. The memory 106 may include a convolutional neural network 108. As discussed herein, the convolutional neural network 108 may be trained using data 110 to classify image data 112 according to predicted patient response to treatment.

The memory 106 may also include modules 114, 116, 118 respectively comprising algorithms/instructions for sorting, preprocessing, segmenting, and classification. A prediction module 122 may use the classification 120 to determine a likely treatment outcome for a patient based on the input pre-treatment biopsy images 124.

The computing device 102 may output the response prediction response 126 at a display device 128, such as a computer monitor. The response prediction output 126 may present a health professional with an estimate (e.g., in a percentage or text format) of how likely a patient is to respond to treatment.

While an embodiment of a system may locally employ a convolutional neural network 108 for the pretreatment prediction of a response to a treatment, other or the same embodiments may perform one or more functions, including sorting, segmenting, and predicting, remotely via a wireless or Internet connection 134. In such a scenario, the input pre-treatment biopsy images 124 may be provided to a remote system that includes convolutional neural network 128 storing the image data 130 and trained data 132.

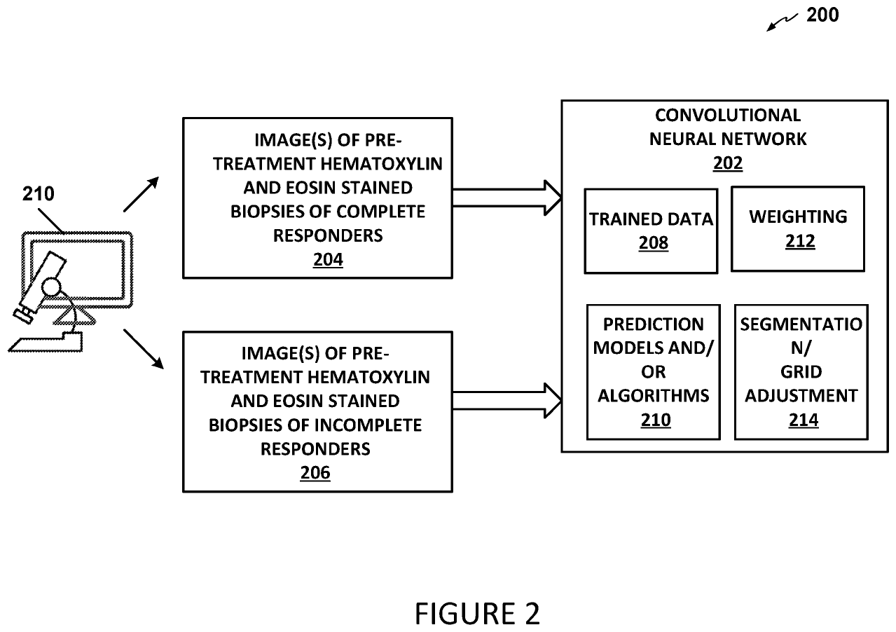

FIG. 2 is block diagram of an embodiment of a system 200 used to train a convolutional neural network 202 to classify image data according to its likelihood of being associated with a patient who will or will not respond to a given therapy. The convolutional neural network 202 may be similar to the convolutional neural networks 108 or 128 of FIG. 1.

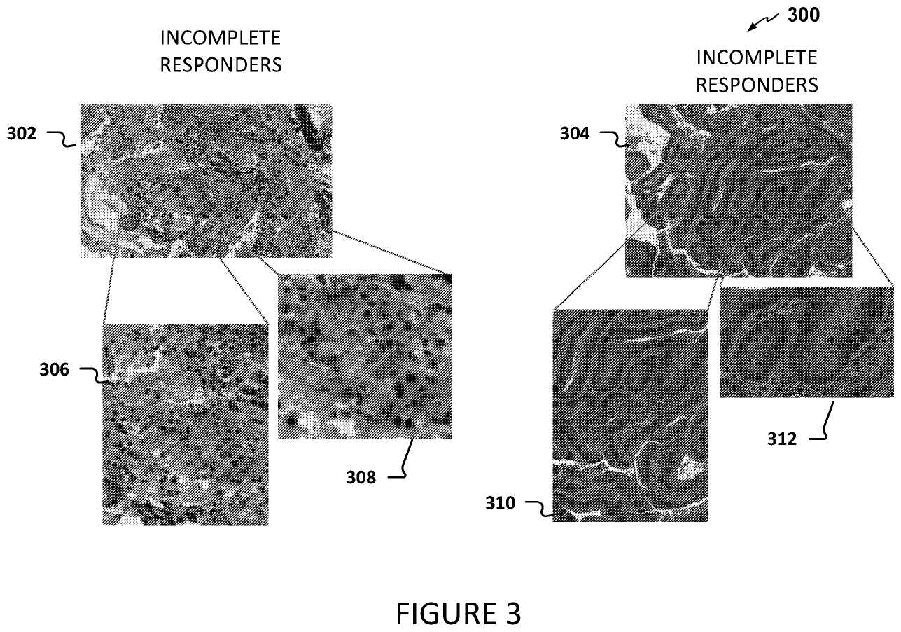

The image data of a particular embodiment of the system may include images of pre-treatment hematoxylin and eosin-stained biopsies of complete responders 204. Other image data may comprise images of pre-treatment hematoxylin and eosin-stained biopsies of incomplete responders 206. The image data 204, 206 may thus be linked to known treatment outcomes for the associated patients. The image data 204, 206 may comprise trained data 208 that is used by prediction algorithms and/or models 210 of the convolutional neural network 202 to continuously learn. To this end, the convolutional neural network 202 may additionally include weighting processes 212 and segmentation processes and grid adjustment 214. FIG. 3 depicts such segmentation and associated grid adjustment processes.

Turning more particularly to FIG. 3, the drawing includes a series of images that include image data 302 associated with complete responders, as well as image data 304 associated with incomplete responders. The image data 302 associated with complete responders is depicted as being segmented into smaller grid images 306, 308. Likewise, the image data 304 associated with incomplete responders is segmented in FIG. 3 into smaller sections, or grids 310, 312. Processing smaller sections of an image may facilitate the speed and efficiency of image processing. The size of the grids may be present or adjusted over time based on training results.

In a particular example, the segmentation processes at a convolutional neural network, such as convolutional neural network 108 or 202 of FIG. 1 or 2, may fine-tune the dimensions of grids based on an accuracy of the treatment predictions over time. As such, an embodiment of the system may be trained with different segmentation scenarios. The results of the scenarios may be correlated with treatment results to determine the accuracy of the predictions. The grid sizes resulting with more accurate predictions may be used to iteratively refine the segmentation processes. As shown in FIG. 3, the grid dimensions may be nonuniform and vary according to particular features of the image to be segmented. For example, preprocessing may identify features of tissue identified in an image that merit a smaller, more focused image analysis.

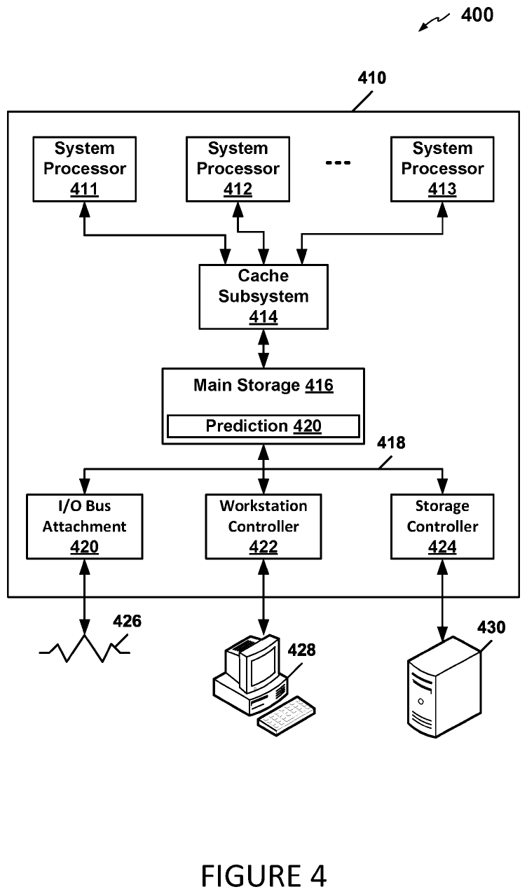

The examples in the drawings demonstrate manners in which program code may use to determine the appropriateness of a treatment protocol (e.g., that includes chemotherapy) for a given patient. For example, FIG. 4 generally illustrates a data processing apparatus 400 configured to train a and assess predictions using image data. The apparatus 400 may generally include a computer, a computer system, a computing device, a server, a disk array, client computing entity, or other programmable device, such as a multi-user computer, a single-user computer, a handheld device, a networked device (including a computer in a cluster configuration), a mobile phone, a video game console (or other gaming system), etc. The apparatus 100 may be referred to as a computing device, such as the computing device 100 of FIG. 1 for the sake of brevity.

The computer 410 generally includes one or more physical processors 411-413 coupled to a memory subsystem including a main storage 416. The main storage 416 may include a flash memory, a hard disk drive, and/or another digital storage medium. As shown in FIG. 1, the main storage 116 may include prediction program code 420 for use in predicting a patient's chance of success with a proposed treatment.

The processors 411-413 may be multithreaded and/or may have multiple cores. A cache subsystem 414 is illustrated as interposed between the processors 411-413 and the main storage 416. The main storage 416 may include logic, or other program code, configured to determine and isolate faulty components. The cache subsystem 414 typically includes one or more levels of data, instruction and/or combination caches, with certain caches either serving individual processors or multiple processors.

The main storage 416 may be coupled to a number of external input/output (I/O) devices via a system bus 418 and a plurality of interface devices, e.g., an I/O bus attachment interface 420, a workstation controller 422, and/or a storage controller 424 that respectively provide external access to one or more external networks 426, one or more workstations 428, and/or one or more storage devices 430, such as a direct access storage device (DASD). The system bus 418 may also be coupled to a user input (not shown) operable by a user of the computer 110 to enter data (i.e., the user input sources may include a mouse, a keyboard, etc.) and a display (not shown) operable to display data from the computer 410 (i.e., the display may be a CRT monitor, an LCD display panel, etc.), and an optical sensor (not shown). The computer 410 may also be configured as a member of a distributed computing environment and communicate with other members of that distributed computing environment through a network 426.

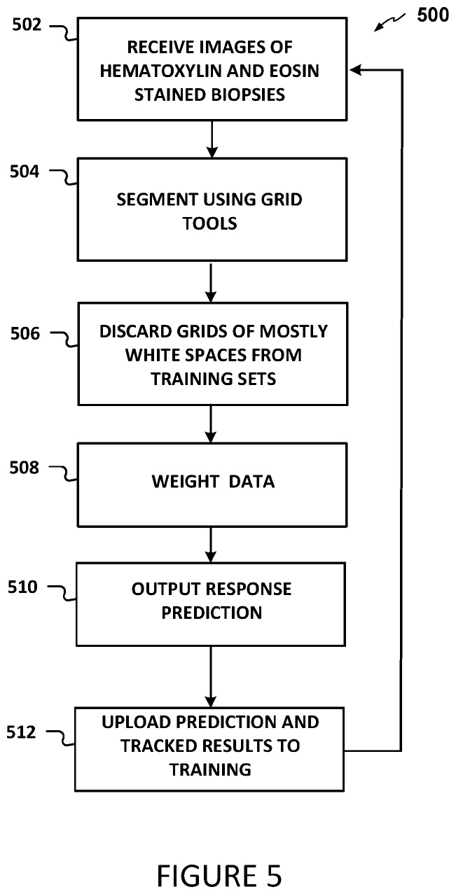

FIG. 5 is a flowchart of an embodiment of a method 500 of predicting use a convolutional neural network of pre-treatment histopathological images to identify patients who will experience a complete response to SCRT and consolidation chemotherapy. The embodiment of the method 500 may be performed by the convolutional neural networks 108 or 128 of FIG. 1, for example.

At 502, the method 500 may include receiving one or more images of treated patient tissue. For example, the computing device 102 of FIG. 1 may receive hematoxylin and eosin-stained biopsies. The hematoxylin and eosin-stained biopsies may be similar to the biopsies 302, 304 illustrated in FIG. 3. The image data received at 502 may not be linked to known patient treatment outcomes. That is, the one or more images may be submitted for purposes of determining a likelihood of a treatment's success for a patent.

At 504, the method 500 may segment the received image data into smaller dimensions using grid tools. For instance, the segmentation at 504 may be performed by the segmentation module 214 of the system 200 of FIG. 2.

The method 500 may discard at 506 grids that are unlikely (e.g., base on analysis of trained, empirical data) to bear fruitful analytical results. For example, the method 500 may discard grids comprising mostly white spaces. The discarding and segmentation may comprise parts of preprocessing such are described in terms of the modules 114, 116, and 118 of FIG. 1. For instance, other preprocessing features may remove whitespace from received or segmented images, while another or the same embodiment may rescale tissue features within an image or segmented image. Other embodiments may flip and rotate the image data to facilitate automatic identification.

Data may be mathematically weighted at 508 as part of refining the training of the prediction processes. For instance, the classifying processes at the convolutional neural network classifier may be fine-tuned using mathematical weighting to predict biological behavior or responsiveness to therapy in rectal cancer. Such weighting may be applied to different features of tissue captured in the images adjusted automatically according to the accuracy of the prediction. The weighting combinations resulting with more accurate predictions may be used to iteratively refine the classification processes by virtue of nodal weights being updated based on training from histopathological slide images.

The method 500 may at 512 output a prediction response. For instance, the prediction response outputted at 512 may be similar to the response prediction output 126 of the system 100 of FIG. 1.

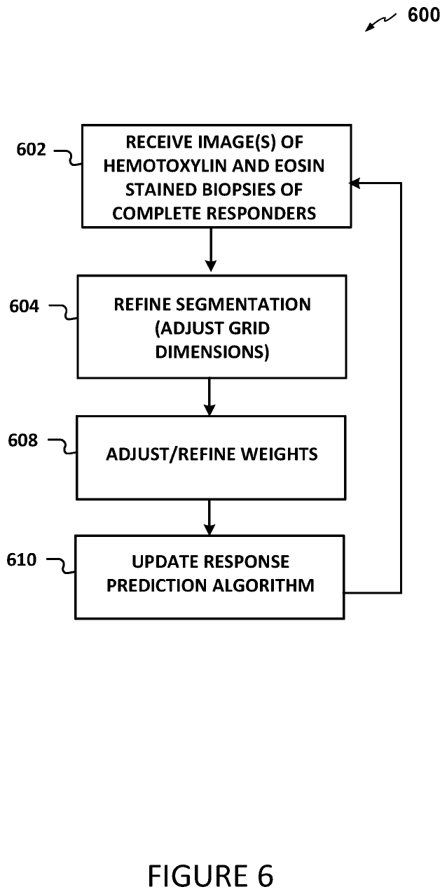

FIG. 6 is a flowchart of an embodiment of a method 600 of training a convolutional neural network to classify image data according to its likelihood of being associated with a patient who will or will not respond to a given therapy. The embodiment of the method 600 may be performed by the convolutional neural network 202 of FIG. 2 and to the convolutional neural networks 108 or 128 of FIG. 1.

At 602, the method 600 may include receiving one or more images of treated patient tissue. For example, the computing device 102 of FIG. 1 may receive hematoxylin and eosin-stained biopsies. The hematoxylin and eosin-stained biopsies may be similar to the biopsies 302, 304 illustrated in FIG. 3. The image data received at 602 may be linked to known patient treatment outcomes for purposes of training the convolutional neural network.

The method 600 at 604 may perform segmentation process and adjust grid dimensions at 604. The method 600 at 606 may at 608 may adjust and refine weights, as described herein. At 610, the method 600 may update the prediction algorithm. For instance, the system 200 may use apply weighting 212 a segmentation algorithm 212, 214, respectively, to the trained data 208.

The method 600 may at 610 update the response prediction algorithm, such as the prediction module 420 of FIG. 4.

While techniques discussed herein are described in greater detail in terms of utilizing a convolutional neural network as a neural network architecture, one skilled in the art will recognize that an embodiment of a method may alternatively or additionally include other types of neural network architectures.

Particular embodiments described herein may take the form of an entirely hardware embodiment, an entirely software embodiment or an embodiment containing both hardware and software elements. In a particular embodiment, the disclosed methods are implemented in software that is embedded in processor readable storage medium and executed by a processor, which includes but is not limited to firmware, resident software, microcode, etc.

Further, embodiments of the present disclosure, such as the one or more embodiments may take the form of a computer program product accessible from a computer-usable or computer-readable storage medium providing program code for use by or in connection with a computer or any instruction execution system. For the purposes of this description, a non-transitory computer-usable or computer-readable storage medium may be any apparatus that may tangibly embody a computer program and that may contain, store, communicate, propagate, or transport the program for use by or in connection with the instruction execution system, apparatus, or device.

In various embodiments, the medium may include an electronic, magnetic, optical, electromagnetic, infrared, or semiconductor system (or apparatus or device) or a propagation medium. Examples of a computer-readable storage medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and digital versatile disk (DVD).

A data processing system suitable for storing and/or executing program code may include at least one processor coupled directly or indirectly to memory elements through a system bus. The memory elements may include local memory employed during actual execution of the program code, bulk storage, and cache memories which provide temporary storage of at least some program code in order to reduce the number of times code must be retrieved from bulk storage during execution.

Input/output or I/O devices (including but not limited to keyboards, displays, pointing devices, etc.) may be coupled to the data processing system either directly or through intervening I/O controllers. Network adapters may also be coupled to the data processing system to enable the data processing system to become coupled to other data processing systems or remote printers or storage devices through intervening private or public networks. Modems, cable modems, and Ethernet cards are just a few of the currently available types of network adapters.

The previous description of the disclosed embodiments is provided to enable any person skilled in the art to make or use the disclosed embodiments. Various modifications to these embodiments will be readily apparent to those skilled in the art, and the generic principles defined herein may be applied to other embodiments without departing from the scope of the disclosure. Thus, the present disclosure is not intended to be limited to the embodiments shown herein but is to be accorded the widest scope possible consistent with the principles and features as defined by the following claims.

1. A pretreatment prediction system comprising:
a display device;
a computing device comprising a processor and a memory, the memory storing instructions that, when executed by the processor, cause the processor to:
receive at least one image of a pre-treatment biopsy from a patient;
process the at least one image using a trained convolutional neural network (CNN);
display, on the display device, a prediction of a response of the patient to a treatment base on a result of the processing of the at least one image using the trained CNN.

2. The pretreatment prediction system of claim 1, wherein the treatment comprises at least one of: radiation, chemotherapy, or immune therapy treatment.

3. The pretreatment prediction system of claim 1, wherein the prediction of the response of the patient to the treatment comprises a likelihood that the patient will experience a complete clinical response to the treatment.

4. The pretreatment prediction system of claim 1, wherein the at least one image of the pretreatment biopsy from the patient comprises at least one stained pre-treatment rectal adenocarcinoma biopsy.

5. The pretreatment prediction system of claim 1, wherein the memory stores the trained CNN.

6. The pretreatment prediction system of claim 1, wherein the trained CNN is stored remotely from the computing device, and the instructions stores in the memory cause the processor to process the at least one image using the trained CNN by transmitting the at least one image for input to the remotely stored CNN.

7. The pretreatment prediction system of claim 1, further comprising selecting a grid size.

8. A method of predicting a treatment outcome, the method comprising:
receiving at least one image of a pre-treatment biopsy from a patient;
processing the at least one image using a trained convolutional neural network (CNN); and
displaying, on a display device, a prediction of a response of the patient to a treatment base on a result of the processing of the at least one image using the trained CNN.

9. The method of claim 8, wherein the treatment comprises at least one of: a radiation, a chemotherapy, or an immune therapy treatment.

10. The method of claim 8, wherein the prediction of the response of the patient to the treatment comprises a likelihood that the patient will experience a complete clinical response to the treatment.

11. The method of claim 8, wherein the at least one image of the pretreatment biopsy from the patient comprises at least on stained pre-treatment rectal adenocarcinoma biopsy.

12. The method of claim 8, wherein the memory stores the trained CNN.

13. The method of claim 8, wherein the trained CNN is stored remotely from the computing device, and the instructions stores in the memory cause the processor to process the at least one image using the trained CNN by transmitting the at least one image for input to the remotely stored CNN.

14. The method of claim 8, further comprising selecting a grid size

15. A computer readable storage medium comprising instructions that when executed by a processor cause the processor to:
receive at least one image of a pre-treatment biopsy from a patient;
process the at least one image using a trained convolutional neural network (CNN); and
display, on a display device, a prediction of a response of the patient to a treatment base on a result of the processing of the at least one image using the trained CNN.

16. The computer readable storage medium of claim 15, wherein the treatment comprises at least one of: a radiation, a chemotherapy, or an immune therapy treatment.

17. The computer readable storage medium of claim 15, wherein the prediction of the response of the patient to the treatment comprises a likelihood that the patient will experience a complete clinical response to the treatment.

18. The computer readable storage medium of claim 15, wherein the at least one image of the pretreatment biopsy from the patient comprises at least on stained pre-treatment rectal adenocarcinoma biopsy.

19. The computer readable storage medium of claim 15, wherein the memory stores the trained CNN.

20. The computer readable storage medium of claim 15, wherein the trained CNN is stored remotely from the computing device, and the instructions stores in the memory cause the processor to process the at least one image using the trained CNN by transmitting the at least one image for input to the remotely stored CNN.
